(19) United States
(12) Patent Application Publication
(10) Pub. No.: US 2011/0004166 A1
(43) Pub. Date: Jan. 6, 2011

(54) DOSE INDICATOR FOR A DEVICE FOR ADMINISTERING MEASURED DOSES OF A LIQUID PRODUCT

(76) Inventors: Juergen Wittmann, Burgdorf (CH); Aurele Horisberger, Burgdorf (CH); Patrick Hostettler, Hasle (CH); Malte Kladiwa, Bern (CH); Stefan Meier, Aarberg (CH); Peter Stettler, Ersigen (CH); Martin Wittwer, Wyssachen (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/794,274

(22) Filed: Jun. 4, 2010

Related U.S. Application Data
(63) Continuation of application No. PCT/CH2008/000517, filed on Dec. 4, 2008.

(30) Foreign Application Priority Data
Dec. 6, 2007 (DE) DE102007058 659.2

(51) Int. Cl. A61M 5/31

(52) U.S. Cl. 604/207

(57) ABSTRACT

A dosage indicator for a device for the metered administration of a dose of a substance, comprising a rotatably movable metering element for adjusting the dose to be administered, an indicator element for indicating the adjusted dose, and a coupling device having a carrying element moveable rotatably about a first axis of rotation and at least one rotatable engagement element disposed on the carrying element for the mechanical coupling of the indicator element and the metering element, wherein the at least one engagement element can be rotated about a second axis of rotation on the carrying element said second axis of rotation extending transversely to the first axis of rotation.

### CROSS-REFERENCED RELATED APPLICATIONS

This application is a continuation of International Patent Application No. PCT/CH2008/000517 filed Dec. 4, 2008, which claims priority to German Patent Application No. DE 10 2007 058 659.2 filed Dec. 6, 2007, the entire contents of each of which are incorporated herein by reference.

### BACKGROUND

The present invention relates to devices for injecting, infusing, administering, delivering or dispensing a substance or product, and to methods of making and using such devices. More particularly, it relates to a device for administering a substance, e.g. a medicinal or therapeutic substance, in selected and/or measured doses or amounts, wherein a selected dose is displayed by the device and/or by a dose indicator associated with the device. The present invention also relates to a process or method of visually displaying a dose of an injectable product, which dose can be selected, on a device for administering the product.

When using administering devices by which a dose of product to be administered can be set or selected, it should be possible to perceive or take a reading of the set or selected dose unequivocally and as easily as possible. Displaying the dose may be particularly useful in situations where a user of the device is not medically trained. There is a whole range of drugs for which self-administration has become commonplace; in self-administration it is the user patient him or her self who administers the relevant drug. Self-administration is common in the treatment of diabetes, for example. Reading a selected or set dose can be problematic, especially if the administering process takes place under cover, in other words is invisible to others, or if the sight of the person undertaking self-administration is impaired. Regardless, however, it is always desirable for the dose to be displayed in a simple manner, unequivocally and so that it can be easily read. This also applies if administration is being undertaken by medically trained staff.

Patent specification EP 0 554 996 B1 discloses a dispensing device with an indicator unit comprising two scale rings. In this instance, the first scale ring is connected to the dose setting element from which the user sets the dose so that it can not rotate. Consequently, when the dose setting element is fully rotated, the first scale ring also rotates by 360°. Every time the first scale ring is fully rotated, the second scale ring is rotated further by one unit.

Since the first scale ring is prevented from rotating relative to the dose setting element, the user must rotate the dose setting element by a full rotation to cause a full rotation of the first scale ring. In the case where a helical spring is provided for applying the dispensing forces when dispensing the liquid product from the administering device, it is also fully rotated when the first scale ring is fully rotated. At a given dose quantity, the helical spring is subjected to stress as a result of the numerous rotations which, ultimately, can limit the service life of the helical spring. It is therefore advantageous to opt for a system whereby a step-down in ratio can be obtained between the dose setting element and first scale ring, which means that the helical spring is rotated a fewer number times for a given dose quantity and a given number of rotations of the scale ring.

Patent specification DE 10 2004 063 652 A1 discloses a device for administering set doses of a liquid product, with a coaxially disposed dose indicator, and the dose indicator also comprises two scale rings. In this instance, the user changes the dose by turning a dose setting element which is connected so that it rotates in unison with a planet carrier of a planetary gear. The planets of the planetary gear roll on an annulus, which is in turn connected so that it rotates in unison with the housing of the dose setting device, and the rotation of the planets causes a rotation of a sun gear connected so that it rotates in unison with the first scale ring. Every full rotation of the first scale ring causes a second scale ring to be rotated by a part-unit. The axis of rotation of the planet gears and the sun gear of the planetary gear extends parallel with the axis of rotation of the dose setting element, planet carrier and scale rings. Due to the step-down in ratio obtained by the planetary gear, the first scale ring can be rotated a higher number of times by a limited number of rotations of the dose setting element. The fact that the planetary gear has to roll on the internal circumference of the annulus and at the same time on the external circumference of the sun gear of the first scale ring means that it is necessary to provide a large amount of radial mounting space, however, which increases the diameter of the administering device as a result.

### SUMMARY

An object of the present invention is to provide an injection device and/or a dose indicator of a compact size, which is nevertheless easy to read and wherein components are subjected to as little stress as possible when setting or selecting the dose.

In one embodiment, the present invention comprises a dosage indicator for a device for the metered administration of a substance, the device comprising a rotatably movable metering element for selecting and/or adjusting the dose to be administered, an indicator element for indicating the selected and/or adjusted dose, and a coupling device having a carrying element moveable rotatably about a first axis of rotation and at least one rotatable engagement element disposed on the carrying element for the mechanical coupling of the indicator element and the metering element, wherein the at least one engagement element can be rotated about a second axis of rotation on the carrying element said second axis of rotation extending transversely to the first axis of rotation.

In one embodiment, the present invention comprises a device for administering an injectable product, comprising a housing with a holder for the product, a dose setting mechanism for setting a dose of the product to be administered and a dispensing mechanism for dispensing the dose. The holder may be an actual container for the product itself or a holder for a container filled with the product, for example an ampoule. The dose setting mechanism enables the dose to be set or selected and/or adjusted, e.g. by rotating a dose setting element which can be operated to set a dose. In principle, however, a dose setting element may also be provided in the form of a push-button or sliding knob or pivoting arm, which sets the dose when repeatedly operated. The dispensing mechanism may have a driving plunger which can be moved axially and serves as a driving element acting directly on the product. In principle, however, the driving element may also be provided in the form of a rotating driving element or peristaltic pumps in a known manner, for example.

In one embodiment, the administering device has a dose indicator with at least one indicator element able to move in rotation. Where another indicator element is provided, the two indicator elements will be referred to hereafter as the first indicator element and second indicator element.

In some embodiments, the indicator element is provided with a dose scale for displaying the dose in scale steps. Accordingly, a mark or indicia is provided for every unit of dose, e.g. a digit.

In accordance with some embodiments of the present invention, the indicator element is mounted so that it can move in rotation about an axis of rotation. When setting the dose, the indicator element is therefore rotated relative to the housing about an axis rotation. In some embodiments, the housing comprises a base or frame, relative to which the indicator element is moved. The frame or housing surrounds the indicator element in some preferred embodiments, although this is not necessarily the case. For example, the indicator element may also be a sleeve structure, which may surround a part of the housing constituting the mounting frame.

In some preferred embodiments, a coupling element may be provided between the indicator element and dose setting element which is moved in rotation to set the dose and couples the movement of the indicator element with the movement of the dose setting element. The coupling operates on the basis of a step-up in ratio so that when the dose setting element is rotated, the indicator element is rotated by a multiple thereof. However, it would also be conceivable to operate on the basis of a step-down in ratio, in which case, to rotate the indicator element, the dose setting element will have to be rotated by a multiple thereof. The indicator element may therefore rotate more quickly or more slowly than the dose setting element by any multiple or fraction. In some preferred embodiments, the indicator element rotates by two times the rotation of the dose setting element so that the indicator element rotates by 720° with every full rotation of the dose setting element. In some preferred embodiments, a differential gear is used as the step-up gear, and in some preferred embodiments a carrier element is coupled with the dose setting element so that it rotates in unison with it. The rolling elements disposed on the carrier element, e.g. in the form of gears, may roll on a fixed base, e.g. a fixed housing, and drive the indicator element. As a result, the indicator element travels a distance which is twice the rotation of the dose setting element, for example, as a result of which the dose to be administered can be set higher for a specific rotation of the dose setting element. The carrier element of the differential gear may incorporate one or more meshing elements or gears, the rotation axes of which extend transversely to the axis of rotation of the carrier element itself. In this respect, the meshing elements need not necessarily be gears, and it would also be possible to use rolling elements which act on elements disposed adjacent to them by friction, for example.

The above-described and other embodiments of the present invention may be advantageous in the case of administering devices where dispensing is operated or powered by a driving spring which is tensed when the dose is being set, and/or in the case of reusable dispensing devices which are fitted with a spring which is tensed when selecting the dose, including in the case of the spring distance of the driving spring being relatively short.

In some embodiments, so that larger doses may be set, another indicator element, in addition to the indicator element described above, may be provided. Like the first indicator element, the second indicator element has a dose scale, e.g. to display a higher dose scale. The user is then able to read the set dose from the dose scales. The second indicator element may be similar to the first indicator elements in terms of shape and size.

In some embodiments, one of the dose scales displays the dose in the smallest dose units which can be set and has a mark, e.g. a digit or other suitable indicia, for every dose unit or a low multiple of the dose unit. The other dose scale is used to display a bigger multiple of the dose unit and its scale steps correspond to a specific multiple of the scale steps of the finer dose scale. The second dose scale is also a scale displaying digits. In some embodiments, the finer dose scale displays units and the scale for higher doses displays tens.

To help ensure that the dose can be clearly read while setting it, in some embodiments, the indicator element is provided with the higher dose scale, which in some preferred embodiments is a scale displaying the tens. Thus, it may be useful for it to be driven so that it is only moved when the dose is changed by the width of the scale steps of the higher scale when setting the dose. When the indicator element with the higher scale is moved back in the direction toward a minimum dose position during dispensing, e.g. the zero dose position, this will also correspond to resetting the dose indicator.

In some preferred embodiments, the indicator elements are reset to an initial state again as a result of dispensing a set or selected dose, so that when the set dose has been completely dispensed, the indicators display the zero dose again or optionally another minimum dose. To achieve this, the indicator elements are operably coupled with the dispensing mechanism during dispensing.

In some embodiments, the dispensing mechanism may incorporate a driving spring, which is tensed during setting when the dose is increased, the absorbed spring energy being released during dispensing, thereby driving or powering the driving element. The dispensing mechanism may be fitted so that the user has to apply some of the force needed for dispensing and the driving spring assists in this process. However, in some preferred embodiments, it is merely necessary to release the driving spring for dispensing and it is the driving spring which provides the force for dispensing on its own once released or triggered. In such embodiments, the dispensing mechanism is released when triggered by a user. One advantage is that, in the case of administering devices where the product is administered by an injection needle, the user does not have to apply additional force in the longitudinal direction of the injection needle for administering purposes after inserting the needle and triggering. In some embodiments, the driving spring may be a spring based on gas pressure or a mechanical spring, for example a compression spring or a torsion spring. A torsion spring may be useful if the dose is selected by a rotating dose setting element and the torsion spring is tensed about the rotation axis of the rotating dose setting element.

In some preferred embodiments, the dose setting element is coupled with the second indicator element by or via the first indicator element. In such embodiments, the flow of force which causes the indicator elements to move when setting the dose runs or travels from the dose setting element via the first indicator element to the second indicator element.

In some embodiments, the indicator elements may be connected to one another via one or more coupling elements to transmit a movement of one of the indicator elements caused by the dose setting element to the other one when the dose is being set so that the latter effects the movement necessary to display the dose.

In some preferred embodiments, the second indicator element is operated by a driving element secured to the first indicator element so that it can not rotate, which is driven at the same time, e.g. moved axially, being activated by the carrier element. This driving action takes place with every full rotation of the first indicator element. The driving action may be generated by a positive connection or friction-based connection, for example by teeth or suitable friction surfaces provided on the relevant elements. Circumferentially extending teeth are provided on the driver element and second indicator element which mesh in one another whenever the second indicator element is operated. For the other rotating movement of the first indicator element, in other words when the second indicator element is not being operated and therefore sits idle in the housing, a catch or lock or stop element secured on the base so that it rotates may be retained by similar or the same type of driving teeth or a friction surface, thereby preventing any unintentional rotation of the second indicator element.

In some preferred embodiments, the dispensing mechanism is uncoupled from the dose setting mechanism when the dose is being set. The dispensing mechanism has an operating element, e.g. a push-button, which can be moved in the longitudinal direction of the administering device or transversely to the longitudinal direction. The operating element is uncoupled from a driving element of the dispensing mechanism when the dose is being set. If the administering device is provided with a driving spring for dispensing purposes, the driving spring and driving element are uncoupled when the dose is being set and coupled with one another for dispensing purposes. In such embodiments, the administering device has a dispensing coupling is uncoupled while the dose is being selected and coupled during dispensing.

In some embodiments, while a dose is being set or during dispensing, the first or second indicator element may be disposed in a force flow of the dispensing mechanism so that it or they transmit the force needed for dispensing to what is ultimately the driving element acting directly on the product. In some preferred embodiments, the indicator elements are disposed outside such a force flow so that they transmit no force to the driving element during dispensing. One or both of the indicator elements may be disposed in a force flow between the dose setting mechanism and the dispensing mechanism when a dose is being set if a force has to be transmitted from the dose setting mechanism to the dispensing mechanism when a dose is being set. However, the indicator elements could be disposed outside of such a force flow, in other words no force is transmitted to a driving element of the dispensing mechanism, including when the dose is being set. This positioning away from the respective flow of force, i.e. away from the flow of force during dispensing or a flow of force which might occur when the dose is being set, gives more design freedom in terms of the shape and disposition of the indicator elements and the dose scales. Another suitable design can then be used for the indicator elements.

In some preferred embodiments, the indicator elements are reset to an initial state again due to dispensing so that they display the zero dose again or another minimum dose which might be different from zero once a set dose has been fully dispensed. To achieve this, the indicator elements are coupled with the dispensing mechanism during dispensing. To hold the indicator elements away from the force flowing to the driving element, as in some preferred embodiments, the flow of force branches inside the dispensing mechanism during dispensing. One branch runs through the entire dispensing mechanism to the driving element and an auxiliary branch runs to the indicator elements. The auxiliary branch may run via the indicator elements or one of the indicator elements to another mechanism of the administering device but, in some preferred embodiments, terminates at one of the indicator elements. In embodiments of this type, the dispensing mechanism has a transmitting mechanism in which the flow of force branches because the transmitting mechanism is coupled with the driving element via a first coupling and with one of the indicator elements via another coupling at its output end.

In some embodiments, it would also be conceivable for the first indicator element to be driven indirectly by the locating elements via a component connected to it. It may also activate a driving element via teeth or a partial toothing with every full rotation, which in turn activates the second indicator element. The locating elements need not necessarily roll on a housing component and instead may also roll on elements connected to the housing so as to rotate in unison with it.

### BRIEF DESCRIPTION OF THE DRAWINGS

FIG. 1 is a longitudinal sectional view of an embodiment of an administering device in accordance with the present invention with a preferred embodiment of a dose indicator in accordance with the invention.

FIG. 2 is a perspective view of the dose indicator.

FIG. 3 is a sectional view of the dose indicator.

FIG. 4 is a perspective view of the dose indicator with indicator elements.

FIG. 5 is a perspective, sectional view showing the dose indicator without indicator elements.

FIG. 6 is a sectional view illustrating another embodiment of the dose indicator.

FIG. 7 is a perspective view of another embodiment of the dose indicator.

FIG. 8 is a perspective view of a portion of another embodiment of the present invention.

### DETAILED DESCRIPTION

With regard to fastening, mounting, attaching or connecting components of the present invention, unless specifically described as otherwise, conventional mechanical fasteners and methods may be used. Other appropriate fastening or attachment methods include adhesives, welding and soldering, the latter including with regard to the electrical system of the invention, if any. In embodiments with electrical features or components, suitable electrical components and circuitry, wires, wireless components, chips, boards, microprocessors, inputs, outputs, displays, control components, etc. may be used. Generally, unless otherwise indicated, the materials for making embodiments of the invention and/or components thereof may be selected from appropriate materials such as metal, metallic alloys, ceramics, plastics, etc. Unless otherwise indicated specifically or by context, positional terms (e.g., up, down, side, front, rear, distal, proximal, etc.) are descriptive not limiting. Same reference numbers are used to denote same parts or components.

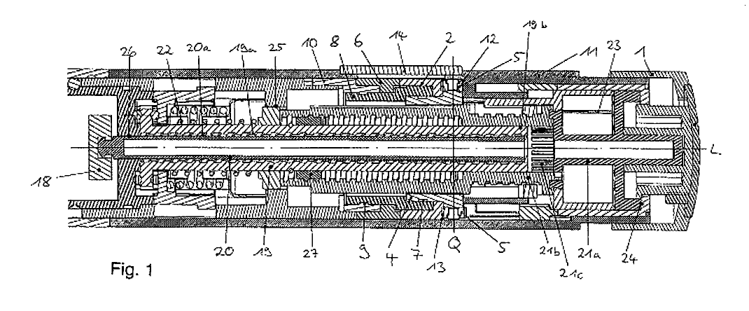

In accordance with the present invention, FIG. 1 is a perspective view of one embodiment of a device for administering an injectable product, for example insulin, a growth hormone or an osteoporosis medicament. The administering device has a proximal (rear) housing part 11, on which a distal (front or forward) housing part, not illustrated, can be fitted so that they can be fixedly and non-detachably connected to one another to form a slim, elongate housing. Such administering devices may be referred to as injection pens because of their size and shape. An ampoule containing the product to be administered may be accommodated in the distal housing part. A needle unit with an injection needle pointing or extending generally along the longitudinal axis of the device can be attached to the part of the housing at the distal end.

At its proximal (rear) end, the administering device has a dose setting element 1, which simultaneously serves as an operating element. The dose setting element 1 is attached to the housing part or also the base 11 so that it can be rotated backward and forward generally about and/or along a central longitudinal axis R of the administering device and can be moved axially in translation relative to the housing part 11. The dose setting element 1, provided in the form of a rotating knob, is used to select, i.e. set, and/or adjust a dose or amount of the product or substance to be administered for each injection. It also acts as a trigger because it can be moved axially along the axis R and causes a set dose to be dispensed when operated axially, i.e. dispensing is caused by depressing the dose setting element 1 in the distal (forward, front, delivery or injection) direction.

To display the set dose, the administering device has an embodiment of a dose indicator in accordance with the present invention. The dose indicator is a two-digit display with a dose scale A2 and a dose scale A6, from which a reading can be taken through a window 14 of the housing part 11. The two dose scales A2 and A6, e.g. the units scale A2 and the tens scale A6, are each composed of a sequence of digits and the figure zero, which are disposed adjacent to one another along the axis L in the illustrated exemplary embodiment. The user can read the set dose comfortably and clearly by holding the administering device with its distal end pointing toward the body, which corresponds to the typical orientation immediately before and during using the device to make an injection.

The dose indicator comprises a first indicator element 2 provided with the dose scale A2 and a second indicator element 6 provided with the dose scale A6. Like the indicator element 6, the indicator element 2 can be rotated backward and forward relative to the housing 2 about and/or along the axis L. The axis L is therefore the rotation axis of the indicator elements 2 and 6. In the depicted exemplary embodiment, the housing or housing part 11 surrounds the two indicator elements 2 and 6 and forms a rotary bearing for them. The dose setting element 1 and the first indicator element 2 are disposed coaxially with respect to the common axis of rotation L and are coupled with one another by the coupling mechanism 3 when the dose is being set so that they can be rotated about the rotation axis R during setting.

The coupling mechanism 3 has a carrier element, which is able to move in rotation about the rotation axis L and four gears 5 disposed circumferentially around the carrier element 4. During a rotating movement of the carrier element 4, the gears 5 roll on circumferentially extending teeth 12 of the housing 11. As they do so, they also drive the first indicator element 2 by their end face by its circumferentially extending teeth 13. In some embodiments, this causes the first indicator element 2 to reach substantially exactly twice the rotation speed of the carrier element 4 connected to the dose setting element 1 so as to rotate in unison with it. The indicator elements 2 and 6 are coupled with one another via the driving element 8. With every full rotation of the indicator element 2, a guide pin (not shown) of the driving element 8 passes through an indentation 7a of a groove 7 in the carrier element 4. As a result, the driving element 8 is pushed axially in the direction of the indentation 7a, in other words in the distal direction opposite the indicator element 6. As this happens, circumferentially extending teeth 15 on the driving element 6 positively mesh in co-operating teeth 17a on the indicator element 6. Consequently, the indicator element 6 is rotated with the indicator element 2 and with the driving element 8 rotationally fixed to it for as long as the indentation 7a is causing the axial displacement of the driving element 8. Having passed the end of the indentation 7a, the driving element 8 is moved back into its original position so that the teeth 15 are disengaged from the teeth 17a. In the subsequent phase, in other words when the guide pin of the driving element 8 is not passing through the indentation 7a, the indicator element 6 is prevented from rotating by other teeth 16 of the catch element 10, which mesh in co-operating teeth 17b of the indicator element 6. The catch element 10 in this instance is connected to the housing 11 so that it is not able to rotate but is able to move axially. It also runs in a groove 9 of the driving element 8. As soon as the driving element 8 is pushed distally, therefore, the catch element 10 is also pushed distally, as a result of which the teeth 16 disengage from the teeth 17b. When the driving element 8 is reset, the teeth 16 therefore mesh with the teeth 17b of the indicator element 6 again, thus preventing the indicator element 6 from rotating.

The indicator elements 2 and 6 are mechanically coupled with one another so that a rotating movement of the indicator element 2 caused by the action of setting the dose causes a rotating movement of the indicator element 6 about the rotation axis L corresponding to the scale steps of the two dose scales A2 and A6.

With a view to enabling a clear and unequivocal display of the dose, in some embodiments the dose scales A2 and A6 move by one scale step when, and only when, the dose is changed by the step width of the relevant dose scale A2 or A6. This is achieved due to the fact, e.g., that the dose scale A6 only moves if the dose is changed by a full 10 units. The set dose corresponds to the pair of digits disposed next to one another in the viewing window 14 of the dose scales A2 and A6. This pair of digits can be read through the viewing window 14 (FIG. 1).

A driving element 18 is disposed in the ampoule (not illustrated). The driving element 18 is a plunger in the embodiment described as an example here, which can be pushed in the ampoule toward the injection needle (not shown) to dispense the product through the injection needle. The stroke by which the driving element 18 will travel with each dispensing operation is determined by the setting of the dose.

The driving element 18 is a constituent part of a dispensing mechanism and is the dispensing mechanism element acting directly on the product. The dispensing mechanism has an output mechanism comprising a first output element 19, a second output element 20 and the driving element 18. The output element 20 acts directly on the driving element 18 and is a plunger rod in the embodiment illustrated and described as an example. The output elements 19 and 20 are connected to one another by a thread.

The second output element 20 has a thread 20a around the rotation axis L, being an external thread in the embodiment illustrated, which establishes a threaded engagement with a co-operating thread 19a of the first output element 19. The first output element 19 is able to rotate relative to the housing 11 about the rotation axis L in a first direction of rotation. The output elements 19 and 20 are able to move in a telescoping action relative to one another. They overlap one another axially. In the embodiment illustrated as an example, the output element 20 extends axially into the output element 19 and can be extracted from the output element 19 during dispensing.

The dispensing mechanism also has a transmitting mechanism (which may be thought of as comprising elements or component mechanisms 21a, 21b) which acts as an input element of the dispensing mechanism in that a driving force causing dispensing is transmitted to the transmitting mechanism 21b. The transmitting mechanism as a whole can be rotated backward and forward generally along and/or about the rotation axis L and can be moved backward and forward axially along the rotation axis L. It is biased in the proximal (rearward) direction by a return spring 22, and the return spring 22 transmits a spring force to the transmitting mechanism 21b acting in the proximal direction. The return spring 22 acts as a compression spring and pushes on the transmitting mechanism in the proximal direction.

A driving spring 23 is supported on the transmitting mechanism 21a. The driving spring 23 generates the driving force because it transmits a spring force to the transmitting mechanism 21a acting in the circumferential direction about the rotation axis L. In the embodiment illustrated as an example, the driving spring 23 is a helical spring with several spring turns wound around the rotation axis L and extending round one another. At one end, the inner end in the depicted exemplary embodiment, it is supported on the transmitting mechanism 21a and its other end, the outer end in the depicted embodiment, is supported on a support connected to or part of the housing 11 to prevent it from rotating. Accordingly, the support is connected to the transmitting mechanism 21a so that it is not able to move axially but the transmitting mechanism 21a is able to move in rotation about the rotation axis L relative to the support.

The transmitting mechanism 21b is connected to the carrier element 4 so that it is prevented from rotating on the rotation axis L but is able to move axially relative to the carrier element 4. In the exemplary embodiment illustrated, the transmitting mechanism 21b and the carrier element engage with one another, preventing relative rotating movements but permitting relative movements in the axial direction. To establish the engagement, at least one axially extending groove and an axially extending rib are provided on mutually facing circumferential faces of the transmitting mechanism 21b and carrier element 4, which mesh one in or with the other. In the illustrated embodiment, several such rotation locks or rotation-locking means are provided on the transmitting mechanism 21b and carrier element 4. The rotation-locking means of the carrier element 4 are disposed on an inner circumferential face of the carrier element 4. The rotation-locking means of the transmitting mechanism 21b for engaging with them are disposed on an outer circumferential face of the transmitting mechanism 21b.

The transmitting mechanism 21 in conjunction with the first output 19 forms a dispensing coupling means. The transmitting mechanism 21b has a dispensing coupling or coupling element 21c and the output element 19 has a dispensing coupling or coupling element 19b, which connect the transmitting mechanism 21b to the output element 19 in the coupled engagement to prevent a rotating movement relative to the rotation axis L but permit an axial relative movement of the transmitting mechanism 21b. The dispensing couplings 21c and 19b are provided in the form of axially extending grooves and locating ribs. In principle, a single groove and a single rib would suffice. The coupled engagement is established for the dispensing operation and maintained during the dispensing operation. When the dose is being set, the dispensing coupling is released so that the entire transmitting mechanism is able to rotate freely relative to the output element 19 when the dose is being selected. The coupled engagement of the dispensing coupling between the dispensing coupling means 21c and 19b is established due to the fact that the transmitting mechanism is moved axially from its illustrated initial position in the distal (forward) direction relative to the output element 19, so that the dispensing coupling 21c moves into the coupled engagement with the dispensing coupling 19b.

An embodiment of the method or way in which an administering device in accordance with the present invention operates will be explained below on the basis of a sequence involved in an exemplary administering of an injection.

The user holds the administering device in the initial state illustrated in FIG. 1 with one hand and sets the desired dose with the other hand by turning the dose setting element 1. During the rotating movement, the dose setting element 4 latches with the housing 11 in a predefined rotary position each time. The first indicator element 2 also rotates together with the transmitting mechanism due to the coupling mechanism 3 but at twice the speed.

Based on the mechanism described above, the indicator element 6 is rotated one unit further with every full rotation of the indicator element 2 by the driving element 8 being connected to the indicator element 2 so that it rotates in unison with it. Through the viewing window 14 in the housing 11, the user reads the dose from the two dose scales A2 and A6 applied to the indicator elements 2 and 6.

As the dose is being set, the driving spring 23 becomes more tightly tensed as the dose is increased. The dose setting mechanism comprising the dose setting element 1 and the transmitting mechanism (comprising elements 21a, 21b) also enables corrections to be made to the dose, e.g. enables the dose to be reduced.

If the dose is corrected, the spring force of the driving spring 23 is reduced. As the dose is being set, the return spring 22 pushes the transmitting mechanism in the proximal (rearward) direction and thus into engagement with the dose setting coupling 24. The transmitting mechanism is free of the first output element 19 in this axial position. In this dose setting position, therefore, rotating movements of the transmitting mechanism are not transmitted to the output element 19 and the elements 20 and 18 of the output mechanism disposed downstream in the drive train. The dose setting mechanism is therefore turned freely by the output mechanism but is still engaged with the dose indicator.

As soon as the user has inserted the injection needle (not illustrated) into the skin, he or she pushes the dose setting element 1 in the distal (forward) direction to initiate dispensing. As it moves axially, the dose setting element 1 pushes the transmitting mechanism in the distal direction relative to the output element 19. During the course of this axial movement, the dispensing coupling means (comprising elements 19b, 21c) is moved into engagement first of all so that the transmitting mechanism and output element 19 are connected to one another to prevent them from rotating. Once the dispensing coupling has engaged, the dose setting coupling 24 is released during the course of the remaining axial movement, followed by the coupling 25, which secures the driving element to prevent it from rotating. The joint triggering movement of the dose setting element 1 and transmitting mechanism terminates as soon as the transmitting mechanism moves into abutment with the output element 19.

Once the dose setting coupling 24 is released, causing the coupled engagement of the dispensing coupling, the driving spring 23 moves the transmitting mechanism in rotation. The transmitting mechanism drives the first output element 19 via the dispensing coupling, i.e. the transmitting mechanism and the output element 19 rotate in unison about the rotation axis L as dispensing now starts. Due to the rotationally locked engagement 26, the second output element 20 is prevented from rotating and turns or screws in the distal direction as a result of the threaded engagement relative to the output element 19. As it moves forward, the output element 20 pushes against the driving element 18 so that it also moves forward in the ampoule causing product to be dispensed through the injection needle. Dispensing is restricted by an anti-rotation stop, against which the transmitting mechanism 21b is moved in the circumferential direction about the rotation axis L.

Since the rotation-locking engagement between the transmitting mechanism 21b and the carrier element 4 also continues to exist during dispensing, the carrier element 4 is turned back into the zero dose position and the indicator elements 6 and 2 are likewise moved into their zero dose position, i.e. the dose indicator A2, A6 is “reset to zero” at the end of a normal dispensing operation.

The injection needle is pulled out of the tissue and the device is ready for a new injection, i.e. for setting another dose and dispensing it.

If the user wishes to set a dose that is higher than the quantity of product still left in the ampoule, this is brought to his or her attention by a stop element 27. In such a situation, the stop element 27 reaches an abutting position and prevents the dose from being increased again. In this case, the dose indicator (comprising elements A2, A6) indicates the quantity of product remaining in the ampoule.

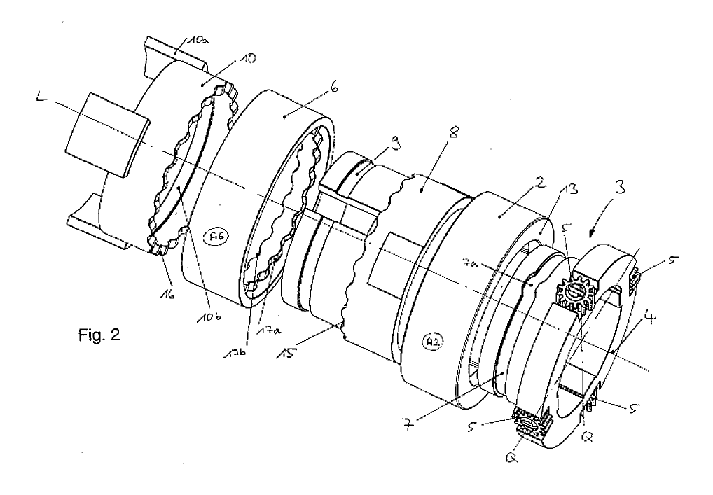

FIG. 2 illustrates one preferred embodiment of a dose indicator in accordance with the present invention. With the exception of the catch element 10, all the elements involved in producing the rotating movement are disposed about the rotation longitudinal axis 11. Only the locating elements 5 also rotate about the rotation axes Q, which are oriented transversely to the rotation axis L. As illustrated, the catch element 10 extends via a circumferentially extending projection 10b into the groove 9 of the driving element 8. Four circumferentially disposed projections 10a which engage in the housing 11 help ensure that the catch element 10 is prevented from rotating but is able to move axially relative to the axis 11.

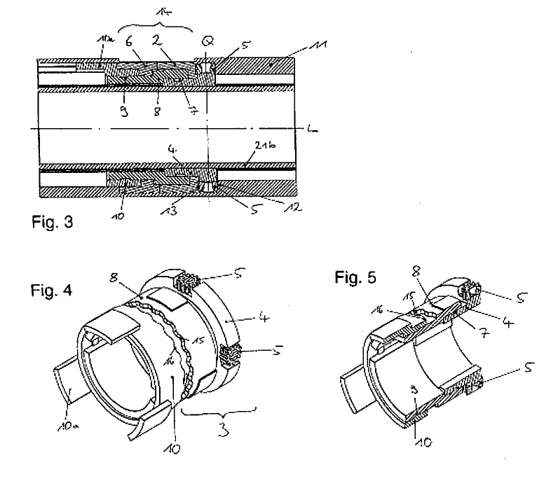

FIG. 3 is a longitudinal sectional view through the dose indicator of FIG. 2, although other elements, e.g. those constituting part of the dispensing mechanism, have been omitted for clarity.

FIGS. 4 and 5 are, respectively, a perspective view and a sectional perspective view of the coupling mechanism 3, which comprises the carrier element 4 and locating elements 5, the driving element 8 and the catch element 10.

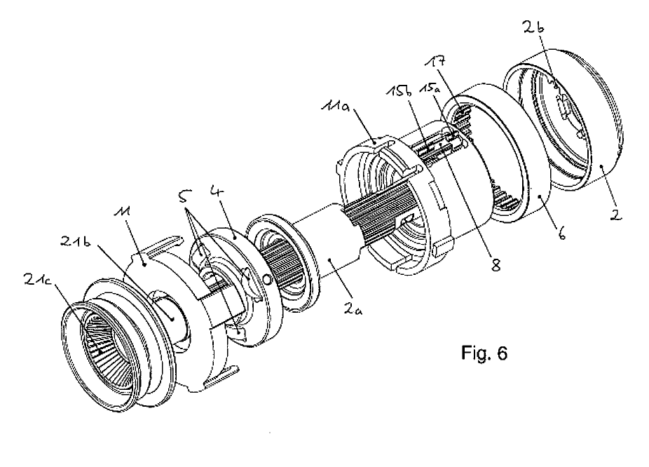

FIG. 6 illustrates another embodiment of the present invention, the function of which will be described below. When a dose is being set, the transmitting mechanism 21b rotates. By teeth 21c, the drive is secured to the transmitting mechanism 21b to prevent rotation. Three locating elements 5 are rotatably mounted on the carrier element 4, perpendicular to the rotation axis of the transmitting mechanism 21b. As soon as the transmitting mechanism 21b rotates, these locating elements 5 roll on the housing or base 11 due to a frictional connection and/or due to suitable texturing or teeth. On the other side, the locating elements 5 engage with the drive 2a of the first indicator element 2. Due to the fact that the rotating movement of the transmitting mechanism 21b is superimposed on the rotating movement of the locating elements 5, the drive 2a rotates twice as fast as the transmitting mechanism 21b. The first indicator element 2 is mounted on the drive 2a so that it is not able to rotate. The rotation of the indicator element 2 causes the partial toothing 2b of the first indicator element 2 to move into engagement with the teeth 15a of the driving element 8 with every rotation of 360°, and the second indicator element 6 engaged with the teeth 15b of the driving element 8 is rotated by the teeth 17 by the distance of the partial toothing 2b.

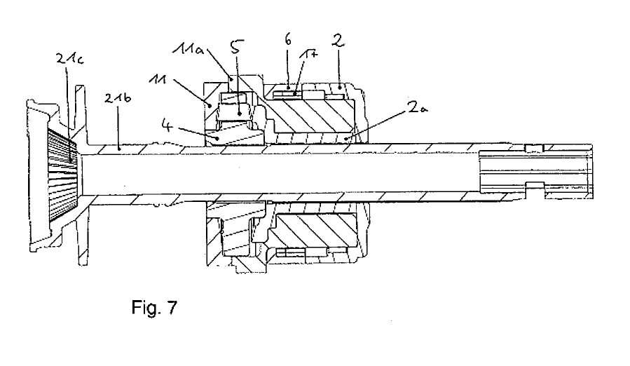

FIG. 7 is a perspective view of the embodiment illustrated in FIG. 6, again in section, in the assembled state. The conical design of the coupling engagement 21c of the transmitting mechanism 21b is illustrated. As also may be seen, the two indicator elements 2, 6 are mounted on the indicator carrier 11a, and the drive 2a of the first indicator element 2 extends through both the second indicator element 6 and the first indicator element 2 and is driven by the locating elements 5.

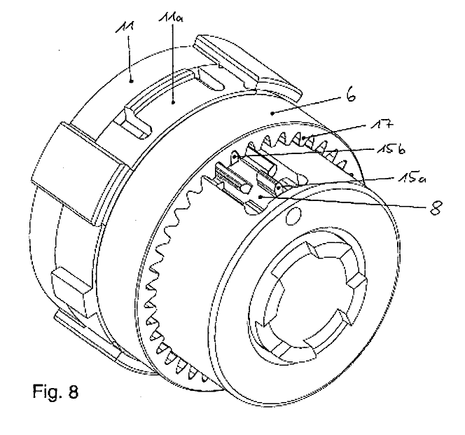

FIG. 8 shows part of the dose indicator of the embodiment illustrated in FIGS. 6 and 7. In this instance, the housing or base 11 is positively connected to the indicator carrier 11a by radially disposed retaining elements and together with it, forms a type of housing which accommodates the carrier element 4, the locating elements 5 and the drive 2a of the first indicator element 2. Also illustrated is the driving element 8, which is disposed on the circumference of the indicator carrier 11a, with its rotation axis disposed at a distance apart from and parallel with the rotation axis of the transmitting mechanism 21b and indicator elements 6 and 2. As also illustrated, the second indicator element 6 is always engaged by its teeth 17 with teeth 15b of the driving element 8. The first indicator element 2 (not visible in this figure) has partial toothing 2c, which “activates” the driving element 8 via the teeth 15a with every full rotation. Accordingly, the driving element 8 rotates by the distance of the partial toothing 2b and drives the second indicator element 6 by the teeth 15b and 17.

Embodiments of the present invention, including preferred embodiments, have been presented for the purpose of illustration and description. They are not intended to be exhaustive or to limit the invention to the precise forms and steps disclosed. The embodiments were chosen and described to illustrate the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A dose indicator for a device for administering set doses of a liquid product, comprising
a) a dose setting element which moves in rotation to set a dose to be administered,
b) an indicator element for displaying the set dose, and
c) a coupling mechanism comprising:
a carrier element which moves about a first axis of rotation, and
at least one locating element disposed on the carrier element which can be moved in rotation to provide a mechanical coupling between the indicator element and the dose setting element, wherein
d) the at least one locating element is able to rotate on the carrier element about a second axis of rotation extending transversely to the first axis of rotation.

2. The dose indicator as claimed in claim 1, further comprising a second indicator element coupled with the first indicator element so that a rotation of the first indicator element causes a partial rotation of the second indicator element.

3. The dose indicator as claimed in claim 1, wherein the indicator element comprises a first and a second annular indicator element.

4. The dose indicator as claimed in claim 2, wherein the second indicator element is driven in rotation by a driving element guided in a radially extending groove of the carrier element and connected to the first indicator element so as to be prevented from rotating and is otherwise retained to prevent rotation by a catch element guided in a radially extending groove of the driving element.

5. The dose indicator as claimed in claim 4, wherein the driving element is able to slide axially and is coupled with the first indicator element so as to rotate in unison with it, and the catch element is coupled with a portion of the device so that it can move axially but is prevented from rotating.

6. The dose indicator as claimed in claim 5, wherein the driving element is moved axially due to a guide pin of the driving element moving through an indentation of the groove of the carrier element, and the driving element moves into engagement with the second indicator element as a result, and the catch element is simultaneously released from the second indicator element.

7. The dose indicator as claimed in claim 6, wherein the driving element and catch element engage with the second indicator element via circumferentially extending teeth on the driving element, catch element and indicator element.

8. The dose indicator as claimed in claim 7, wherein the catch element, driving element and carrier element extend through at least part of the indicator elements.

9. The dose indicator as claimed in claim 1, wherein the carrier element is coupled with the dose setting element so that a rotation of the dose setting element causes a rotation of the carrier element.

10. The dose indicator as claimed in claim 1, wherein at least one locating element is a gear and meshes in circumferentially extending meshing teeth on a portion of the device and in output teeth extending around the circumference of the first indicator element.

11. The dose indicator as claimed in claim 10, wherein the meshing teeth and output teeth are crown teeth and the at least one locating element comprises spur teeth.

12. The dose indicator as claimed in claim 2, wherein each of the outer circumferential surfaces of the indicator elements bears a scale comprising indicia for displaying the set dose which can be viewed through a viewing window of the device.

13. The dose indicator as claimed in claim 1, wherein four locating elements are disposed around the circumference of the carrier element.

14. The dose indicator as claimed in claim 2, wherein the indicator elements are secured axially with respect to the axis of rotation.

15. The dose indicator as claimed in claim 12, wherein one scale is a scale displaying units and the second scale is a scale displaying tens.

16. The dose indicator as claimed in claim 2, wherein the indicator elements are disposed adjacent to one another.

17. The dose indicator as claimed in claim 1, wherein the indicator element is driven by the locating element indirectly via a drive, and the drive extends through the indicator element.

18. The dose indicator as claimed in claim 4, further comprising another driving element which has an axis of rotation spaced at a distance apart from and parallel with the axis of rotation of the carrier element.

19. A device for administering set doses of a liquid product with a dose indicator comprising a dose setting element, an indicator element for displaying a set dose, and a coupling mechanism comprising a carrier element which moves about a first axis of rotation and at least one locating element disposed on the carrier element which can be moved in rotation to provide a mechanical coupling between the indicator element and the dose setting element, wherein the at least one locating element is able to rotate on the carrier element about a second axis of rotation extending transversely to the first axis of rotation, the device further comprising:
a) a housing with a holder for the product,
b) a dose setting mechanism for setting the dose of a product, and
c) a dispensing mechanism for dispensing the dose.

20. The device as claimed in claim 19, wherein the dose setting mechanism comprises a dose setting element which can be operated to set the dose, which is coupled with the indicator element during setting so that operation of the dose setting element causes a movement of the indicator element which is faster than the movement of the dose setting element, and the indicator element is coupled with another indicator element via a driving element so that the movement of one of the indicator elements causes a movement of the other.

21. The device as claimed in claim 20, further comprising a rotating driving spring coupled with the dose setting mechanism when the dose is being set and with the first indicator element during dispensing, whereby the driving spring tensed by increasing the set dose when the dose is being set and one of the indicator elements moves back into a zero dose position during dispensing.

22. The device as claimed in claim 21, further comprising a driving spring coupled with the dose setting mechanism when the dose is being set and with the dispensing mechanism when the dose is being dispensed, whereby the driving spring is tensed by an increase in the dose during setting and drives the dispensing mechanism during dispensing.

23. The device as claimed in claim 22, wherein the dispensing mechanism comprises a transmitting mechanism for absorbing the spring force of the driving spring, an output mechanism and a dispensing coupling by which the transmitting mechanism is coupled with the output mechanism during dispensing.

24. The device as claimed in claim 21, wherein the dose setting mechanism comprises a dose setting element which can be activated, a transmitting mechanism absorbing the spring force of the driving spring and a dose setting coupling by which the dose setting element is coupled with the transmitting mechanism for setting the dose.

25. The device as claimed in claim 24, wherein:
the dose setting coupling connects the dose setting element to the transmitting mechanism in a coupled engagement which prevents rotation,
the dispensing coupling connects the transmitting mechanism to the output mechanism in a coupled engagement which prevents rotation,
the transmitting mechanism is able to move in rotation about an axis of rotation to set the dose and is able to move axially for dispensing,
the dispensing coupling is released when the dose is being set, and
the dose setting coupling is released during dispensing.

26. The device as claimed in claim 25, wherein the indicator elements are coupled with the dispensing mechanism during dispensing and the coupling mechanism causes the indicator elements to be moved into a reset position during dispensing.

27. The device as claimed in claim 26, further comprising an operating element which can be activated for the dispensing operation, an output mechanism with a driving element driving the product, and a transmitting mechanism which transmits at least some of the driving force to the output mechanism during dispensing, wherein the transmitting mechanism is coupled with an indicator element during dispensing to move the indicator elements back into an initial state during dispensing.

28. The device as claimed in claim 20, wherein the dispensing mechanism comprises an operating element, an output mechanism with a driving element driving the product, and a transmitting mechanism which transmits at least some of a driving force to the output mechanism during dispensing, wherein the transmitting mechanism is coupled with one of the indicator elements during dispensing to move the indicator elements back into an initial state during dispensing.

29. The device as claimed in claim 28, wherein the transmitting mechanism has a driving end for transmitting the driving force and an output end, and a flow of force generated by the driving force splits into a first force branching to the output mechanism and a second force branching to the indicator elements at the output end, and the indicator elements are disposed outside of the force flowing to the driving element.
